SEQUENCE LISTING (10 sequences)

SEQ ID NO: 1 - protein, 14 aa, Artificial Sequence
  Patient 10, PBMC 1
CASSTAGAGYEQYF

SEQ ID NO: 2 - protein, 16 aa, Artificial Sequence
  Patient 10, PBMC 2
CASSLGGLAANYEQYF

SEQ ID NO: 3 - protein, 14 aa, Artificial Sequence
  Patient 10, PBMC 3
CASSLAVQGGGYTF

SEQ ID NO: 4 - protein, 16 aa, Artificial Sequence
  Patient 10, PBMC 4
CASSGGGLAGNSEQYF

SEQ ID NO: 5 - protein, 14 aa, Artificial Sequence
  Patient 10, PBMC 5
CASTLGQGAWPLHF

SEQ ID NO: 6 - protein, 18 aa, Artificial Sequence
  Patient 10, opT 1
CATSRQDRGRRWNTEAFF

SEQ ID NO: 7 - protein, 15 aa, Artificial Sequence
  Patient 10, opT 2
CASSVRPTDTDTQYF

SEQ ID NO: 8 - protein, 16 aa, Artificial Sequence
  Patient 10, opT 3
CASSLGEAGGTGELFF

SEQ ID NO: 9 - protein, 14 aa, Artificial Sequence
  Patient 10, opT 4
CASSTAGAGYEQYF

SEQ ID NO: 10 - protein, 17 aa, Artificial Sequence
  Patient 10, opT 5
CASSQLREGGITGELFF

(19) United States
(12) Patent Application Publication
(10) Pub. No.: US 2022/0229043 A1
(43) Pub. Date: Jul. 21, 2022

(54) METHODS FOR GENERATION OF TUMOR ORGANOID-FISHED T CELLS AND IDENTIFICATION OF ANTI-TUMOR T CELL RECEPTORS

(71) Applicant: Beth Israel Deaconess Medical Center, Inc., Boston, MA (US)

(72) Inventors: Manuel Hidalgo, Boston, MA (US); Senthil K. Muthuswamy, Boston, MA (US)

(21) Appl. No.: 17/595,634

(22) PCT Filed: May 29, 2020
(86) PCT No.: PCT/US2020/035099

§ 371 Date: Nov. 19, 2021

Related U.S. Application Data
(60) Provisional application No. 62/855,826, filed on May 31, 2019.

(51) Int. Cl. G01N 33/50; C12N 5/09; C12N 5/0783; C12Q 1/6881

(52) U.S. Cl. CPC G01N 33/505; C12N 5/0693; C12N 5/0636; C12Q 1/6881; C12N 2502/30; C12N 2506/11; C12N 2501/2302; C12N 2501/2315; C12N 2501/2321; C12N 2501/2307; C12N 2501/33; C12N 2502/1114

(57) ABSTRACT

Provided herein are methods for obtaining tumor-targeting T cells, as well as for identifying tumor-targeting TCRs.

### CLAIM OF PRIORITY

This application claims the benefit of U.S. Patent Application Ser. No. 62/855,826, filed on May 31, 2019. The entire contents of the foregoing are hereby incorporated by reference.

### TECHNICAL FIELD

Described herein are methods for the preparation of lymphocytes (e.g., T cells) capable of targeting a tumor, as well as for identification and use of tumor-targeting T cell receptors.

### BACKGROUND

Advances in the understanding of cancer immunobiology and the critical role of the tumor microenvironment in facilitating immune escape and disease growth have opened a new horizon in cancer therapeutics.

### SUMMARY

Provided herein are methods for the preparation of lymphocytes (e.g., T cells) capable of targeting a tumor. The methods include providing cells from a tumor in a subject, and preparing a tumor organoid from the cells; providing lymphocytes from the subject; maintaining the tumor organoid and the lymphocytes from the subject in culture under conditions and for a time sufficient for the lymphocytes to bind to the organoid; separating the tumor organoid and a tumor organoid-bound lymphocyte from unbound lymphocytes; and isolating the tumor organoid-bound lymphocyte.

In some embodiments, preparing the tumor organoid comprises providing a sample comprising tumor tissue from a subject; enzymatically digesting the tissue; plating single cells suspended in media comprising serum-free supplements, fibroblast growth factor, and insulin; and incubating the cells for 2-3 days until a tumor organoid forms.

In some embodiments, the lymphocytes are incubated with IL-2, IL-15, and IL-21 prior to being cultured with the tumor organoid.

In some embodiments, the tumor organoid and the lymphocytes are maintained together in culture for at least 2 hours, up to 6, 12, 24, 48 or 72 hours.

In some embodiments, the methods include expanding the tumor organoid-bound lymphocytes in media comprising IL-7, IL-2, IL-15, and IL-21.

In some embodiments, the methods include administering the isolated and expanded tumor organoid-bound lymphocytes to the subject.

In some embodiments, the methods include identifying one or more tumor-targeting T cell receptor sequences of one or more tumor-targeting T cell receptors expressed on the isolated and expanded tumor organoid-bound lymphocyte. In some embodiments, the methods include expressing the one or more tumor-targeting T cell receptors in a T cell, e.g., a T cell from the subject.

In some embodiments, the methods include administering the T cell expressing the one or more tumor-targeting T cell receptors to the subject.

In some embodiments, the tumor is from pancreatic, breast, liver, lung, ovary, head and neck, glioblastoma or colon cancer.

In some embodiments, separating the tumor organoid and the tumor organoid-bound lymphocyte comprises filtering and/or affinity purification.

Also provided herein are methods for identifying a tumor-targeting T cell receptor. The methods include providing cells from a tumor in a subject, and preparing a tumor organoid from the cells; providing lymphocytes from the subject; maintaining the tumor organoid and the lymphocytes from the subject in culture under conditions and for a time sufficient for lymphocyte-mediated killing of tumor organoid cells to occur; isolating lymphocytes from the culture; sequencing one or more tumor-targeting T cell receptor sequences in the lymphocytes after culturing with the tumor organoid; and identifying the one or more tumor-targeting T cell receptor sequences.

In some embodiments, preparing a tumor organoid comprises providing a sample comprising tumor tissue; enzymatically digesting the tissue; plating single cells suspended in media comprising serum-free supplements, fibroblast growth factor, and insulin; and incubating for 2-3 days until a tumor organoid forms.

In some embodiments, the lymphocytes are incubated with IL-2, IL-15, and IL-21 prior to being cultured with the tumor organoid.

In some embodiments, the methods include maintaining the tumor organoid and the lymphocytes in culture for at least 3, 4, or 5 days. In some embodiments, the tumor organoid and the lymphocytes are maintained in culture for at least 5 (e.g., 5-10) days.

In some embodiments, the methods include expressing the one or more tumor-targeting T cell receptors in a T cell, e.g., a T cell from the subject.

In some embodiments, the methods include administering the T cell expressing the one or more tumor-targeting T cell receptors to the subject.

In some embodiments, the tumor is pancreatic, breast, liver, lung, ovary, head and neck, glioblastoma, or colon cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a representative sample of PMBC expanded cells.

FIG. 2 is a representative system to enrich and purify T cells bound to the organoids (organoid-fished T cells, ofT cells).

FIG. 3 is a representative analysis of ofT cells showing that 94.7% were CD4 T cells.

FIG. 4 is a representative analysis of ofT cells showing that 55% were central memory T cells (TCM).

FIG. 5 is a representative analysis of ofT cells showing that 99.3% were CD95 positive.

FIG. 6A is a pie chart of TCR clones in PBMC.

FIG. 6B is a pie chart of opT cells.

FIGS. 7A-E are a series of photographs illustrating generation of organoid-fished T (ofT) cells.

FIG. 8 is a bar graph showing that expanded ofT are effective in killing tumor organoids.

FIGS. 9A-B are each pairs of graphs showing that ofT respond to autologous tumor cells by proliferating.

### DETAILED DESCRIPTION

The development of immunotherapeutic strategies to reverse immune paralysis, expand tumor-specific effector cells, and create immune memory responses to prevent disease recurrence has begun to yield stunning clinical results. The applicability of immunotherapies to cancers like pancreatic ductal adenocarcinoma (PDAC) represents a paradigm shift in providing a highly innovative and promising therapy to potentially prevent disease recurrence in this highly lethal disease. PDAC tumors are thought to lack tumor-targeting T cells. Provided herein are methods for obtaining tumor-targeting T cells, as well as for identifying tumor-targeting TCRs.

Organoid-Fished T Cells (OfT)

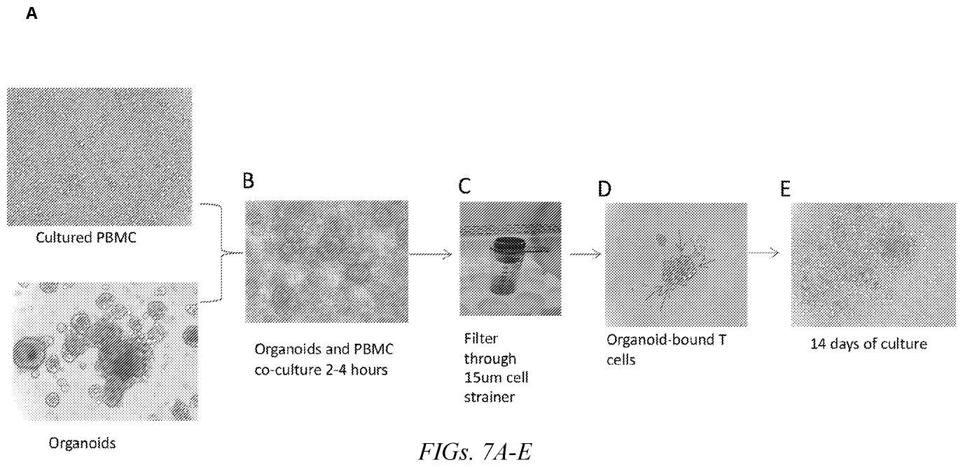

To obtain tumor targeting T lymphocytes, a cancer patient's tumor cells are expanded to establish patient-derived tumor organoid cultures. The tumor-derived organoids are then incubated with cultured PBMC for about 2 hours. T cells bound to the tumor organoids are “fished out,” e.g., by passing the organoid-T cells mixture through a filter with a pore size about 15 μm. T cells bound to tumor organoids are subsequently expanded in the presence of a cytokine cocktail, e.g., comprising IL7, IL2, IL15, and IL21, to generate organoid-fished T cells (ofT). These ofT cells are enriched for CD4+ and CD8+ T cells that express markers of activation (CD95) and central memory. See FIGS. 7A-E.

Purified ofT cells with memory phenotype can be used in multiple applications by 1) re-injecting purified ofT cells back into the patient for adoptive T cell therapy to eliminate the tumor; 2) identifying and using tumor-specific TCRs on ofT cells for adoptive T cell therapy; 3) using ofT cells as a screening platform to identify the best immunotherapy combinations for personalizing immune approaches for patients; 4) using ofT cells to find neoantigens; and 5) identifying new immune checkpoint inhibitors.

In some embodiments, the present methods include a number of important aspects: 1) an interleukin cocktail (IL7; IL2; IL15; IL21) to expand T cells from PBMC; 2) conditions to combine patient's T cells with his/her own tumor organoids to fish-out tumor-targeting T cells with memory phenotype from peripheral blood; and 3) a method to expand organoid-fished T cells with memory phenotype.

Tumor Organoids

The tumor organoids used in the methods described herein can be obtained and prepared using methods known in the art, e.g., as described in WO2016015158 and PCT/US2019/067274, both of which are incorporated herein by reference. For example, the methods can include obtaining a sample comprising tumor tissue, enzymatically digesting the tissue (e.g., using collagenase) and plating single cell suspensions in a biomatrix hydrogel support, e.g., a basement membrane extract such as MATRIGEL, PATHCLEAR Grade Basement Membrane Extract (Amsbio) or other synthetic alternatives, e.g., as described in Nguyen et al., Nat Biomed Eng. 2017; 1. pii: 0096, and maintained in media containing Dulbecco's Modified Eagle Media (DMEM) with factors including serum-free supplements, fibroblast growth factors (FGFs), and insulin, e.g., the Pancreatic Progenitor and Tumor Organoid Media described in WO2016015158.

In some embodiments, the tumor cells used to grow organoids are obtained from a subject who will be treated using a method described herein; in some embodiments, the tumor cells are obtained from a different subject who has a cancer, e.g., of the same type as the subject who will be treated.

Peripheral Blood Mononuclear Cells (PBMC)

The PBMC used in the methods described herein can be obtained and prepared using methods known in the art. For example, obtain 10 ml heparinized blood from patients and centrifuge to remove plasma. The blood will be layered on top of Ficoll to separate PBMCs. PBMC will be cultured in T cell Medium Cellgro with human AB serum, IL-2, IL-15, IL-21 and Amphotericin B to generate tens of millions of PBMC.

### Identification of Tumor-Targeting T Cell Receptors

To promote T-cell mediated killing, a new and emerging strategy uses genetically engineered T cells where a patient's T cells are engineered to express tumor-targeting TCR a and b chains. Initial clinical trials using genetically modified TCR therapies are showing promise, for example, TCR directed against a melanoma antigen (MART1) was cloned from tumor-infiltrating T cells and used to treat patients with melanoma. These studies show feasibility, lack of adverse effects and respectable tumor regression. The success of this approach depends on the ability to identify tumor-specific TCRs.

The current method involves expansion of the rare tumor-infiltrating T cells (TILs) and determining the TCR sequences. TILs are not readily available for all tumors and the TCRs from TILs do not always have the ability to target the tumor. In addition, this methodology is only useful for patients whose tumor is surgically resected which is a minority of cancer patients. The present methods use patient tumor-derived organoid cultures and the patient's own peripheral blood mononuclear cells derived T cells to enrich and identify TCR sequences that have the ability to target tumor cells. These TCR can then be expressed in engineered T cells and re-introduced to the subject, to treat the cancer in the subject. See, e.g., Miliotou and Papadopoulou, Curr Pharm Biotechnol. 2018; 19(1):5-18 and Fiens et al., Am J Hematol. 2019 May; 94(S1):S3-S9, which describe generally methods for use in engineering T cells (e.g., for Chimeric antigen receptor (CAR) T-cell therapy).

Methods of Treatment

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. In some embodiments, the disorder is a solid tumor, e.g., breast, prostate, pancreatic, brain, hepatic, lung, kidney, skin, or colon cancer. Generally, the methods include administering a therapeutically effective amount of a treatment as described herein, e.g., a treatment comprising ofT cells or T cells engineered to express a TCR identified by a method described herein, to a subject who is in need of, or who has been determined to be in need of, such treatment. In some embodiments, the methods include administering a therapeutically effective amount of a treatment comprising a checkpoint inhibitor, a treatment comprising an agent that increases levels of interferons and a checkpoint inhibitor, and/or a standard treatment comprising chemotherapy, radiotherapy, and/or resection. These standard treatments can also be administered in combination with an immunotherapy.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Immunotherapy

In some embodiments, the present methods include the administration of an immunotherapy.

In some embodiments, the immunotherapies primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (8-[4-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013). In another example, M2 macrophage targeted therapy includes clodronate-liposomes (Zeisberger, et al., Br J Cancer. 95:272-281 (2006)), and M2 macrophage targeted pro-apoptotic peptides (Cieslewicz, et al., PNAS. 110(40): 15919-15924 (2013)). Immnotherapies that target Natural Killer T (NKT) cells can also be used, e.g., that support type I NKT over type II NKT (e.g., CD1d type I agonist ligands) or that inhibit the immune-suppressive functions of NKT, e.g., that antagonize TGF-beta or neutralize CD1d.

Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4:734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g., fresolimumab, Infliximab, Lerdelimumab, or GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012)). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, or tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin). See Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-A/B (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3:e28811). Immunotherapies can also include administering one or more of: cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Krüger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4): 342-7.

### EXAMPLES

The invention is further described in the following examples, which do not limit the scope of the invention described in the claims.

### Methods

The following methods were used in the Examples below.

### Expansion of PBMC

1) 10 ml heparin blood from patients.

### 2) Isolate PBMC

3) Culture the PBMC on 24 well plates, 0.3-1 million per well with T cell medium. T cell Medium is Cellgro with 10% human AB serum, containing IL-2, IL7, IL-15, IL-21, Penicillin-Streptomycin and Amphotericin B.
4) Change medium when needed. Keep the cells growing in 1-2 million/ml and passage them for 2-3 weeks.

### Generation of Organoids

5) Establish Pancreatic Tumor Organoid using protocol described herein.
6) Culture Organoids with 1 ml/well PaTOM Growth Media on 12 well plate for 5-7 days.
7) When the organoids ready to use, aspirate the old media
8) Digest the Matrigel and release the organoids using collagenase/dispase and gently pellet the organoids
Generation and Expansion of Organoids Primed T Cells (opT)
9) Incubate organoids with PBMC in one well on 96 well plate with 200 ul T cell medium for 7-10 days
10) After T cell kill all tumor cells, transfer all cells one well on 24 well plate.
11) Stimulated T cell with organoids one more time.
12) After second time stimulation harvest the T cells named organoids primed T cells (opT).

### Identification of TCR in opT Cells.

13) Extracted DNA from 1 million opT cell by QIAamp DNA Blood Mini Kit
14) Deep sequence the TCRVB by Adaptive Biotechnologies ImmunoSEQ. 100,000 to 220,000 T cell were sequenced.

### Example 1. Organoid-Fished T Cells (OfT)

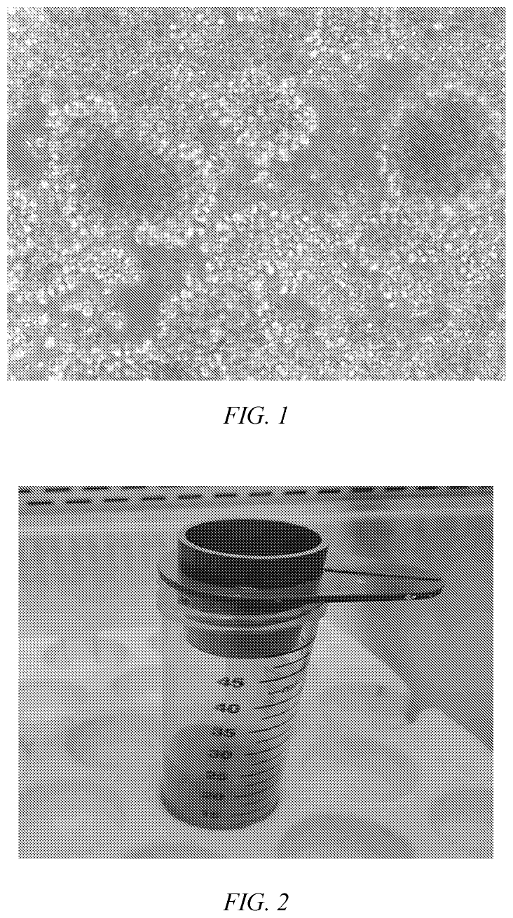

Tumor-targeting T cells from a patient's peripheral blood monocytes cultures (PBMC) were enriched by culturing PMBCs using a media cocktail containing IL2, IL7, IL15, and IL21, which allows expansion of tens of millions of T cells (FIG. 1).

The patient's tumor cells were expanded to establish patient-derived tumor organoid cultures. Tumor tissue was enzymatically digested, suspended in Matrigel, and incubated in media containing serum-free supplements, fibroblast growth factor, and insulin until tumor organoids formed. The tumor-derived organoids were incubated with cultured PBMC for 2 hours. T cells bound to the tumor organoids were “fished out” by passing the organoid-T cells mixture through a filter with a pore size about 15 μm (FIG. 2).

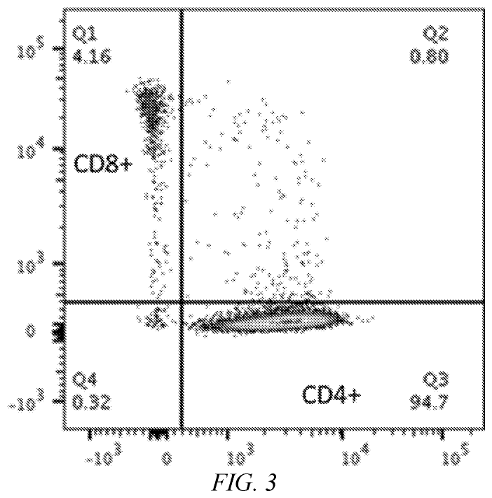

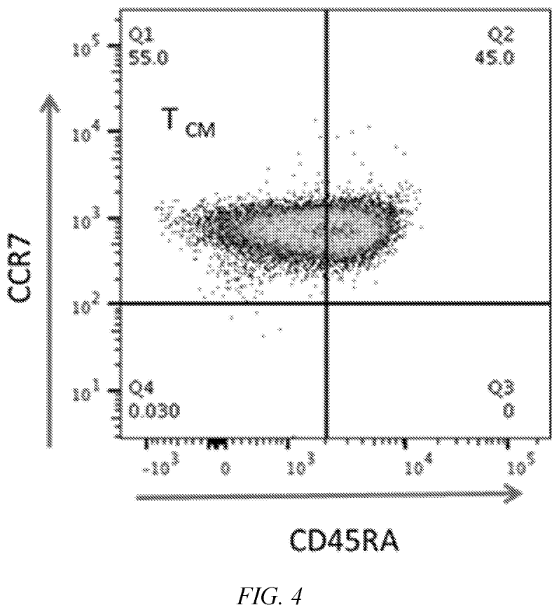

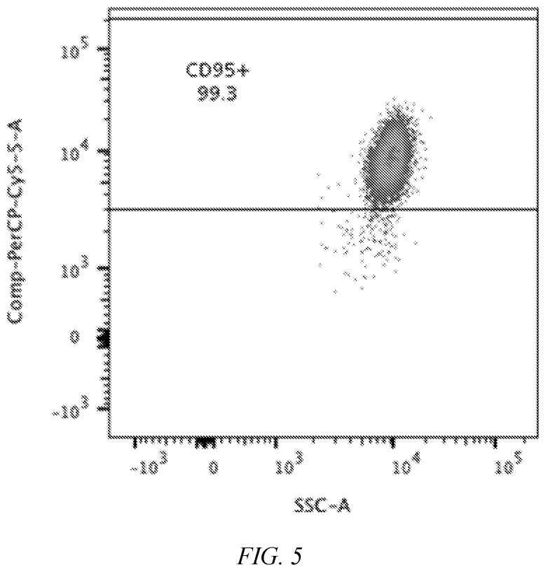

T cells bound to tumor organoids were subsequently expanded in the presence of IL7, IL2, IL15, and IL21 to generate organoid-fished T cells (ofT). These ofT cells were enriched for CD4+ and CD8+ T cells that express markers of activation (CD95) and central memory (FIGS. 3, 4, and 5).

### Example 2. Identification of Tumor-Targeting T Cell Receptors

Tumor-targeting T cells from the patient's peripheral blood monocytes cultures (PBMC) and patient-derived tumor organoid cultures were established as described herein. The tumor-derived organoids were incubated with cultured PBMC for 7-10 days. Optionally, tumor-targeting T cells were stimulated again with tumor organoids after most if not all tumor cells are killed by the tumor-targeting T cells in the first round. Organoid primed T cells (opT) are harvested after the first or second simulation.

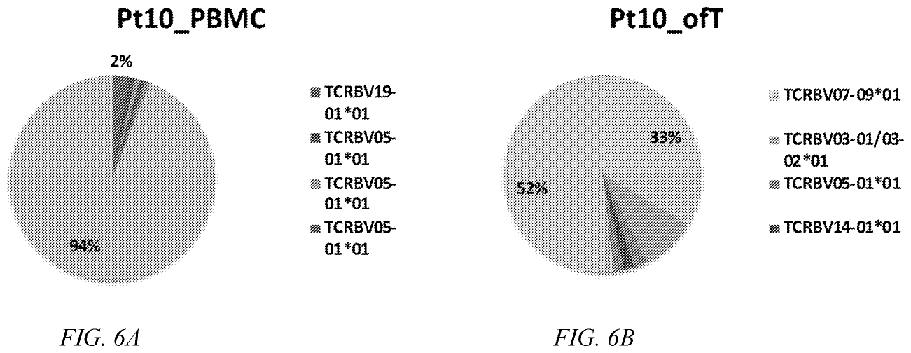

We determined if the ofT cells with cytoxic abilities are a generically activated populations of PBMC or if they have undergone clonal-selection during the ofT generation process. DNA was extracted from opT cells and sequenced to identify enriched TCR sequences. Beta-chains of approximately 100,000 T cell receptor (TCR) from PBMC and opT cells were sequenced. In PBMC-primed samples, the most abundant TCR sequence represented 2.6% of the all the TCRs analyzed (FIG. 6A and Table 1). The rest of the TCRs had less abundance in the population with the top five most abundant TCR clones representing 2.0% of the total TCRs sequenced (FIG. 6A and Table 1). This is consistent with PBMC being made-up of a highly polyclonal T cell population. Interestingly, in ofT the most abundant TCR represented 33.6% of the all the TCR analyzed (FIG. 6B and Table 1). Furthermore, the top five TCRs made-up 40% of all the TCRs present in the population demonstrating a dramatic clonal selection in ofT cells (FIG. 6B and Table 1).

TABLE 1
Top 5 TCRs Sequence detail in PBMC and opT

| | | Fre- | | | | | | Top5 |
|---|---|---|---|---|---|---|---|---|
| | Top | quency | | | | | | Sequence |
| Sample | 5 | (%) | Sequence | # | Vβ | D | J | sum |
| Patient | 1 | 2.6 | CASSTAGAGYEQYF | 1 | TCRBV19- | TCRBD01- | TCRBJ02- | 6.0 |
| 10, | | | | | 01*01 | 01*01 | 07*01 | |
| PBMC | 2 | 1.0 | CASSLGGLAANYE | 2 | TCRBV05- | TCRBD02- | TCRBJ02- | |
| | | | QYF | | 01*01 | 01 | 07*01 | |
| | 3 | 0.8 | CASSLAVQGGGYTF | 3 | TCRBV05- | TCRBD01- | TCRBJ01- | |
| | | | | | 01*01 | 01*01 | 02*01 | |
| | 4 | 0.8 | CASSGGGLAGNSE | 4 | TCRBV05- | TCRBD02- | TCRBJ02- | |
| | | | QYF | | 01*01 | 01 | 07*01 | |
| | 5 | 0.8 | CASTLGQGAWPLHF | 5 | TCRBV06- | TCRBD01- | TCRBJ01- | |
| | | | | | 05*01 | 01*01 | 06*01 | |
| Patient | 1 | 49.1 | CATSRQDRGRRWNTE | 6 | TCRBV15- | TCRBD01- | TCRBJ01- | 89.8 |
| 10, | | | AFF | | 01*02 | 01*01 | 01*01 | |
| opT | 2 | 19.5 | CASSVRPTDTDTQYF | 7 | TCRBV13- | unknown | TCRBJ02- | |
| | | | | | 01*01 | | 03*01 | |
| | 3 | 12.6 | CASSLGEAGGTGE | 8 | TCRBV07- | TCRBD02- | TCRBJ02- | |
| | | | LFF | | 06*01 | 01*02 | 02*01 | |
| | 4 | 7.8 | CASSTAGAGYEQYF | 9 | TCRBV19- | TCRBD01- | TCRBJ02- | |
| | | | | | 01*01 | 01*01 | 07*01 | |
| | 5 | 0.7 | CASSQLREGGITGE | 10 | TCRBV06- | TCRBD02- | TCRBJ02- | |
| | | | LFF | | 06*04 | 01*02 | 02*01 | |

#, SEQ ID NO:

Identification of TCRs specific on tumor-targeting T cells enriched in the opT cell population can be applied to 1) identifying patient-specific anti-tumor TCR sequences that can be used for personalized TCR therapy by engineering his/her T cells to express tumor-targeting TCRs; and 2) identifying pan-cancer targeting TCRs that can be used for engineering any cancer patient's T cell for TCR therapy.

### Example 3. Generation of Organoid-Bound T Cells

This example demonstrates that the organoid-bound T cells undergo clonal selection of specific T cell clones and are effective in killing autologous tumors compared the matched peripheral blood mononuclear cells.

Generation of organoid-fished T (ofT) cells is illustrated in FIGS. 7A-E. As shown in FIG. 7A, tumor organoids dislodged from Matrigel were incubated with peripheral blood derived mononuclear cells from the same patient for 3-4 hours (7B). The mixture of organoids and PBMC were passed through a 10 micron filter so that all unbound PBMC can be washed away and separated from the organoid-bound T cells (7C). After four washes, the organoids that were retained on the filter were transferred to a culture dish (7D, see arrows). The organoids and bound T cells were cultured in the organoid lymphocyte media for two weeks (7E), during which time, the T cells expand and kill tumor organoids in the process. As the tumor-targeting T cell from the circulation are fished-out using organoid, we refer to these T cells as organoid-fished T (ofT) cells.

### Example 4. Expanded OfT are Effective in Killing Tumor Organoid

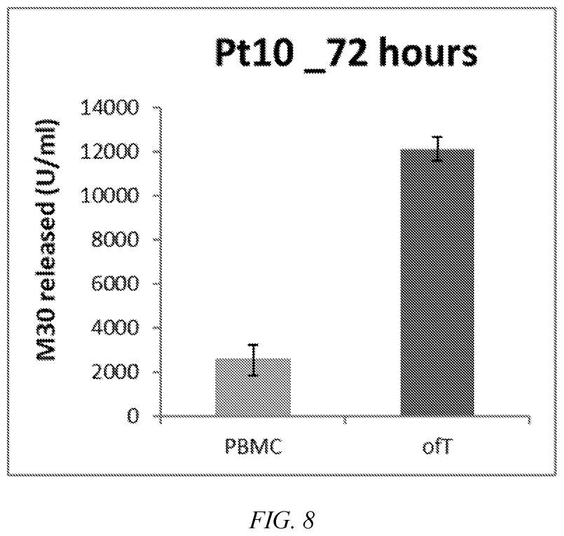

After the ofT cells were generated over a period of 14 days (FIGS. 7A-E and above), the ofT cells were expanded in lymphocyte media for an additional week. We next determined if the ofT cells were better than PBMC in their ability to killed autologous tumor organoids. About 10,000 organoids were seed in each well of a 96 well plate and grown for four days. On Day four, 100,000 PBMC or ofT were added to the well and culture continued for 72 hours. At the end of the experiment, media was collected and analyzed for the presence of cytokeratin fragments released by dying tumor epithelial cells using the M30 assay kit. The results, shown in FIG. 8, demonstrate that ofT cells are six-fold more potent in killing tumor epithelia than matched PBMC demonstrating that ofT cells are effective in their cytotoxic properties.

### Example 5. OfT Respond to Autologous Tumor Cells by Proliferating

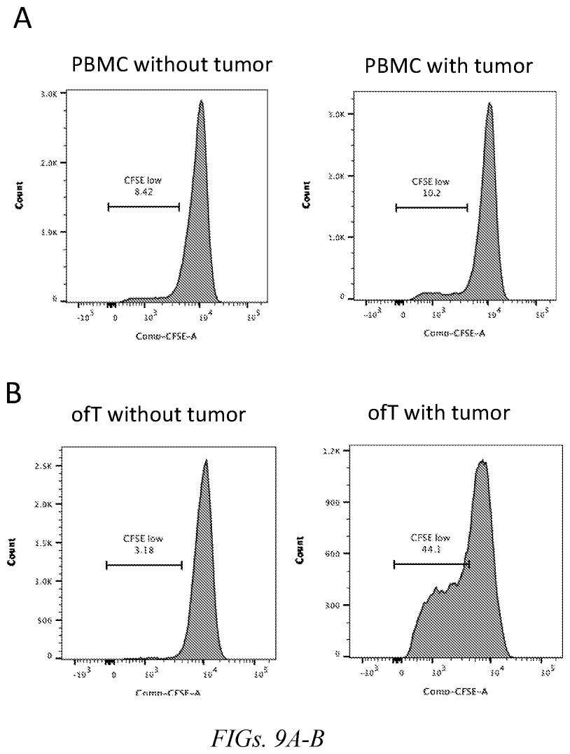

T cells respond to antigen recognition by entering cell cycle to expand in cell number, a property that ensures elimination of the target by its cytotoxic activity. We determined if PBMC and ofT cells differ in their ability to respond to tumor organoids. About 10,000 organoids were seed in each well of a 96 well plate and grown for four days. On Day four, 100,000 PBMC or ofT cells that were labelled with CFSE were added to the well and culture continued for additional four days. As cells divide, the CFSE labelled is equally divided between the cell progenies, thus, presence of immune cells with progressively lower amount of CFSE signal is an indication of cell division. As shown in the FIGS. 9A-B, the PBMC grown with tumor organoids showed a 2.0% increase the low CFSE populations (9A), whereas ofT cells showed a 41% increase in low-CFSE T cells (9B), demonstrating that ofT cells are more than 20-fold efficient in responding to autologous tumors by entering cell cycle.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the preparation of lymphocytes capable of targeting a tumor, a method comprising:
providing cells from a tumor in a subject, and preparing a tumor organoid from the cells;
providing lymphocytes from the subject;
maintaining the tumor organoid and the lymphocytes from the subject in culture under conditions and for a time sufficient for the lymphocytes to bind to the organoid;
separating the tumor organoid and a tumor organoid-bound lymphocyte from unbound lymphocytes; and
isolating the tumor organoid-bound lymphocyte.

2. The method of claim 1, wherein preparing the tumor organoid comprises:
providing a sample comprising tumor tissue;
enzymatically digesting the tissue;
plating single cells suspended in media comprising serum-free supplements, fibroblast growth factor, and insulin; and
incubating the cells for 2-3 days until a tumor organoid forms.

3. The method of claim 1, wherein the lymphocytes are incubated with IL-2, IL-15, and IL 21 prior to being cultured with the tumor organoid.

4. The method of claim 1, wherein the tumor organoid and the lymphocytes are maintained together in culture for at least 2 hours.

5. The method of claim 1, further comprising expanding the tumor organoid-bound lymphocytes in media comprising IL-7, IL-2, IL-15, and IL-21.

6. The method of claim 5, further comprising administering the isolated and expanded tumor organoid-bound lymphocytes to the subject.

7. The method of claim 5, further comprising identifying one or more tumor-targeting T cell receptor sequences of one or more tumor-targeting T cell receptors expressed on the isolated and expanded tumor organoid-bound lymphocyte.

8. The method of claim 7, further comprising expressing the one or more tumor-targeting T cell receptors in a T cell.

9. The method of claim 8, further comprising administering the T cell expressing the one or more tumor-targeting T cell receptors to the subject.

10. The method of claim 1, wherein the tumor is from pancreatic, breast, liver, lung, ovary, head and neck, glioblastoma or colon cancer.

11. The method of claim 1, wherein separating the tumor organoid and the tumor organoid-bound lymphocyte comprises filtering and/or affinity purification.

12. A method for identifying a tumor-targeting T cell receptor, the method comprising:
providing cells from a tumor in a subject, and preparing a tumor organoid from the cells;
providing lymphocytes from the subject;
maintaining the tumor organoid and the lymphocytes from the subject in culture under conditions and for a time sufficient for lymphocyte-mediated killing of tumor organoid cells to occur;
isolating lymphocytes from the culture;
sequencing one or more tumor-targeting T cell receptor sequences in the lymphocytes after culturing with the tumor organoid; and
identifying the one or more tumor-targeting T cell receptor sequences.

13. The method of claim 12, wherein preparing a tumor organoid comprises:
providing a sample comprising tumor tissue;
enzymatically digesting the tissue;
plating single cells suspended in media comprising serum-free supplements, fibroblast growth factor, and insulin; and
incubating for 2-3 days until a tumor organoid forms.

14. The method of claim 12, wherein the lymphocytes are incubated with IL-2, IL-15, and IL-21 prior to being cultured with the tumor organoid.

15. The method of claim 12, further comprising maintaining the tumor organoid and the lymphocytes in culture for at least 3, 4, or 5 days.

16. The method of claim 12, wherein the tumor organoid and the lymphocytes are maintained in culture for at least 5 days.

17. The method of claim 12, further comprising expressing the one or more tumor-targeting T cell receptors in a T cell.

18. The method of claim 17, further comprising administering the T cell expressing the one or more tumor-targeting T cell receptors to the subject.

19. The method of claim 12, wherein the tumor is pancreatic, breast, liver, lung, ovary, head and neck, glioblastoma, or colon cancer.

20. The method of claim 8, comprising expressing the one or more tumor-targeting T cell receptors in a T cell from the subject.
